(19) United States
(12) Patent Application Publication
(10) Pub. No.: US 2014/0147422 A1
(43) Pub. Date: May 29, 2014

(54) METHOD OF DERIVING STEM CELLS, STEM CELLS, AND USE OF STEM CELLS FOR WOUND HEALING

(71) Applicants: Vance D. Fiegel, Shakopee, MN (US); David R. Knighton, Richmond, MN (US)

(72) Inventors: Vance D. Fiegel, Shakopee, MN (US); David R. Knighton, Richmond, MN (US)

(73) Assignee: Embro Corporation, St. Louis Park, MN (US)

(21) Appl. No.: 14/171,186

(22) Filed: Feb. 3, 2014

Related U.S. Application Data
(63) Continuation of application No. 13/628,924, filed on Sep. 27, 2012, now abandoned.

(51) Int. Cl. C12N 5/074; A61K 35/44

(52) U.S. Cl. CPC C12N 5/0607; A61K 35/44
USPC 424/93.7; 435/377; 435/325; 435/366; 435/354; 435/353; 435/352

(57) ABSTRACT

A method of deriving isolated stem cells including: implanting a matrix in a wound site of a living organism; allowing cells to infiltrate the matrix; removing the matrix containing the infiltrated cells from the wound site; and removing the infiltrated cells from the matrix to provide isolated stem cells. Stem cells produced by this process, stem cells with certain characteristics, and methods for treating wounds using these stem cells are provided.

### FIELD OF THE INVENTION

This invention relates to methods for deriving stem cells, stem cells, and the use of stem cells to heal wounds.

### BACKGROUND OF THE INVENTION

The cellular pathway in wound repair results in regeneration in the mammalian fetus and in species such as newts and starfish. Scarring is the end point of wound repair in most other adult animals. Stem cells are currently used to enhance normal healing but a role for stem cells in the healing cascade is currently unknown. Cellular signaling pathways that lead to regeneration versus scarring with a common stem cell progenitor could explain this difference.

We have previously studied the sequence and progress of wound angiogenesis in vivo and the initiation of wound healing angiogenesis, the role of the wound space microenvironment in wound angiogenesis, the effect of oxygen tension of macrophage angiogenesis, and the role of platelets in wound angiogenesis. Phillips et al., Initiation and pattern of angiogenesis in wound healing in the rat, Am J Anat 1991, 192:257-262. Knighton et al., Role of platelets and fibrin in the healing sequence: An in vivo study of angiogenesis and collagen synthesis, Ann Surg 1982, 196:379-388. Knighton et al., Regulation of wound healing angiogenesis. Effect of oxygen gradients and inspired oxygen concentration, Surgery 1981, 90:262-270. Knighton et al., Oxygen tension regulates the expression of angiogenesis factor by macrophages, Science 1983, 221:1283 1285. Michaeli et al., The role of platelets in wound healing: Demonstration of angiogenic activity, In: Hunt T K, et al. editors, Soft and Hard Tissue Repair: Biological and Clinical Aspects, New York: Praeger Publishers, 1984:380-394.

The wound healing response is a complex and intricate interaction between inflammatory cells and the specific cell types involved in the actual repair process. Singer et al., Cutaneous wound healing, New England J Med 1999, 341: 738-746. The primary cells involved in repair, endothelial cells, fibroblasts, keratinocytes, and nerve cells, comprise all three germ layers.

Stem cells are used for the treatment of various diseases. Embryonic stems cells have drawn the most attention due to their innate ability to theoretically differentiate into any type of terminal cell. Ying et al., The ground state of embryonic stem cell self-renewal, Nature 2008, 453:519-523. For this reason, embryonic stem cells have long been the “holy grail” of stem cell therapeutics. They have been induced to become a number of various terminal tissue cells and used in a large number of potential therapeutic applications. Lerou et al., Therapeutic potential of embryonic stem cells, Blood Rev 2005, 19:321-331.

Adult stem cells have been identified and isolated from a variety of tissues. Stem cells have long been known to exist in bone marrow and have been used clinically for decades. More recently, adult stem cells have been isolated from a number of different tissue sources including adipose tissue, umbilical cord, and multi-potent adult progenitor cells from bone marrow. Zuk, The adipose-derived stem cell: looking back and looking ahead, Mol Biol Cell 2010, 21:1783-1787. Malgieri et al., Bone marrow and umbilical blood mesenchymal stem cells: state of the art, Int J Clin Exp Med 2010, 3:248-269. Herdrich et al., Multipotent adult progenitor cells: their role in wound healing and the treatment of dermal wounds, Cytotherapy 2008, 10:543-550. Reprogrammed stem cells (iPS) are actually terminal cells into which the active genes characteristic of undifferentiated stem cells have been introduced to cause the cells to appear to de-differentiate to a stem cell like status. Das, Induced pluripotent stem cells (iPSCs): the emergence of a new champion in stem cell technology-driven biomedical applications, J Tissue Eng Regen Med 2010, 4:413-421. Stadtfeld et al., Induced pluripotency: history, mechanisms, and applications, Genes Dev 2010, 24:2239-2263. Jopling et al., Dedifferentiation, transdifferentiation and reprogramming: three routes to regeneration, Nature Rev Molec Cell Biol 2011, 12:79-89. Additional studies have also demonstrated the ability of transcription factors to directly convert terminally differentiated cells into functional cells of another lineage. Jopling et al., Dedifferentiation, transdifferentiation and reprogramming: three routes to regeneration, Nature Rev Molec Cell Biol 2011, 12:79-89. Vierbuchen et al., Direct conversion of fibroblasts to functional neurons by defined factors, Nature 2010, 463:1035-1041.

We investigated wound derived capillary endothelial cells (WCEC) to determine if they possessed stem cell properties.

### SUMMARY OF THE INVENTION

The invention provides a method of deriving isolated stem cells comprising: implanting a matrix in a wound site of a living organism; allowing cells to infiltrate the matrix; removing the matrix containing the infiltrated cells from the wound site; and removing the infiltrated cells from the matrix to provide isolated stem cells.

The invention provides isolated stem cells derived from the methods described herein.

The invention provides isolated stem cells that are positive for Oct-4 and SSEA-1 as determined by immunofluorescence staining and have measurable telomerase levels while control fibroblasts have no measurable telomerase levels as determined by an enzyme-linked immunosorbent assay.

The invention provides a method of treating wounds comprising: implanting a matrix in a first wound site of a first living organism; allowing cells to infiltrate the matrix; removing the matrix containing the infiltrated cells from the wound site; removing the infiltrated cells from the matrix to provide isolated stem cells; and applying the isolated stem cells to a second wound site that is on the first or a second living organism.

The invention provides a method of deriving, culturing, and differentiating isolated stem cells comprising: implanting a matrix in a wound site of a living organism; allowing cells to infiltrate the matrix; removing the matrix containing the infiltrated cells from the wound site; removing the infiltrated cells from the matrix to provide isolated stem cells; culturing the isolated stem cells in an undifferentiated state; and subsequently differentiating the isolated stem cells into a specific cell type.

It is to be understood that both the foregoing general description and the following detailed description are exemplary and explanatory and are intended to provide further explanation of the invention as claimed.

### BRIEF DESCRIPTION OF THE DRAWINGS

FIG. 1 shows staining of WCEC for acetylated-LDL uptake. WCEC were isolated from sponges and placed into culture. The left side of FIG. 1 shows a phase contrast image of WCEC cultured on a MATRIGEL-coated surface. The right side of FIG. 1 shows the same field of view as the left side under fluorescence microscopy demonstrating the specific uptake of fluorescently labeled acetyl-LDL by WCEC.

FIG. 2 shows the chemotactic response of WCEC to various isoforms of PDGF. Chemotactic assays were performed as described in the Materials and Methods. PDGF isoforms were tested from 0.01-100 ng/ml and cell migration is expressed as cells per high power field.

FIG. 3 shows the expression of PDGF receptors by WCEC. Various concentrations of ¹²⁵I-PDGF-BB from 0.5-10 ng/ml were added to WCEC for receptor binding studies. Non-specific binding was determined in the presence of a 100-fold excess of unlabelled PDGF-BB. Data was analyzed according to the method of Scatchard for the determination of receptor Kd and the number of receptors/cell.

FIGS. 4 and 5 show the ability of TGFβ to induce WCEC to form capillary structures in vitro. WCEC were suspended into collagen gels and incubated for 5 days at 37° C. in the presence (FIG. 5) or absence (FIG. 4) of 0.5 ng/ml TGFβ. Following incubation, frozen sections were made and examined by Nomarski interference contrast microscopy.

FIG. 6 shows the ability of PDGF-BB to induce receptor mediated phosphorylation in WCEC. WCEC and 3T3 fibroblasts were examined for PDGF-BB and basic FGF induced receptor tyrosine phosphorylation. The lanes are as follows: Lane 1—phosphorylated PDGF receptor control, Lane 2—untreated WCEC, Lane 3—WCEC treated with 1 ng/ml PDGF-BB, Lane 4—WCEC treated with 1 ng/ml basic FGF, Lane 5—untreated 3T3 fibroblasts, Lane 6—3T3 fibroblasts treated with 1 ng/ml PDGF-BB, and Lane 7—3T3 fibroblasts treated with 1 ng/ml basic FGF.

FIGS. 7 and 8 show the staining of WDSC for Oct-4 expression. WDSC were isolated from sponges and placed into culture on MATRIGEL-coated flasks. FIG. 7 shows a phase contrast image of WDSC. FIG. 8 shows the same field of view as FIG. 7 under fluorescence microscopy demonstrating staining for Oct-4.

FIGS. 9 and 10 show the staining of WDSC for SSEA-1 expression. WDSC were isolated from sponges and placed into culture on MATRIGEL-coated flasks. FIG. 9 shows a phase contrast image of WDSC. FIG. 10 shows the same field of view as FIG. 9 under fluorescence microscopy demonstrating staining for Oct-4.

FIGS. 11 and 12 show the induction of osteoblast formation in WDSC. WDSC were cultured in the presence or absence of osteoblast differentiation media for nine days and stained for mineralization with alizarin red. FIG. 11 shows a phase contrast image of untreated WDSC stained with alizarin red. FIG. 12 shows a phase contrast image of WDSC treated with differentiation media and stained with alizarin red demonstrating mineralization (red stain).

FIGS. 13 and 14 show the induction of neuronal cell formation in WDSC. WDSC and a neuronal stem cell were treated with neuronal differentiation media for seven days and stained for nestin. FIG. 13 shows neuronal stem cells demonstrating nestin staining following differentiation. FIG. 14 shows WDSC demonstrating nestin staining following differentiation.

FIG. 15 shows the histological quality of tendon tissue repair without stem cells and with stem cells.

FIG. 16 shows the increase in tensile modulus using stem cells for tendon repair.

FIG. 17 shows the increase in ultimate tensile strength using stem cells for tendon repair.

### DETAILED DESCRIPTION

The invention provides a method of deriving isolated stem cells comprising: implanting a matrix in a wound site of a living organism; allowing cells to infiltrate the matrix; removing the matrix containing the infiltrated cells from the wound site; and removing the infiltrated cells from the matrix to provide isolated stem cells. The living organism may be a mammal, a human, a mouse, a rabbit, or a rat. The matrix may be an open cell sponge. The sponge may be made of polyurethane. The sponge needs to provide a scaffold for the entering cells to infiltrate.

In one embodiment, the isolated stem cells are positive for Oct-4 and SSEA-1 as determined by immunofluorescence staining. In an embodiment, the isolated stem cells are negative for SSEA-3, SSEA-4, Tra-60, and Tra-80 as determined by immunofluorescence staining. In an embodiment, the isolated stem cells have measurable telomerase levels while control fibroblasts have no measurable telomerase levels as determined by an enzyme-linked immunosorbent assay. In an embodiment, the isolated stem cells respond to TGFβ to form capillary-like structures.

In one embodiment, the isolated stem cells are positive for Oct-4 and SSEA-1 as determined by immunofluorescence staining and have measurable telomerase levels while control fibroblasts have no measurable telomerase levels as determined by an enzyme-linked immunosorbent assay. In an embodiment, the isolated stem cells are negative for SSEA-3, SSEA-4, Tra-60, and Tra-80 as determined by immunofluorescence staining. In an embodiment, the isolated stem cells respond to TGFβ to form capillary-like structures. In an embodiment, the isolated stem cells do not respond to acidic or basic FGF in a chemotaxis assay but do respond to PDGF-BB in a chemotaxis assay.

In an embodiment, the method further comprises culturing the isolated stem cells. In one embodiment, the method further comprises purifying the isolated stem cells.

The invention provides isolated stem cells derived from the methods described herein. In an embodiment, the isolated stem cells are positive for Oct-4 and SSEA-1 as determined by immunofluorescence staining and have measurable telomerase levels while control fibroblasts have no measurable telomerase levels as determined by an enzyme-linked immunosorbent assay. In one embodiment, the isolated stem cells are negative for SSEA-3, SSEA-4, Tra-60, and Tra-80 as determined by immunofluorescence staining. In an embodiment, the isolated stem cells respond to TGFβ to form capillary-like structures. In an embodiment, the isolated stem cells do not respond to acidic or basic FGF in a chemotaxis assay but do respond to PDGF-BB in a chemotaxis assay.

The invention provides isolated stem cells that are positive for Oct-4 and SSEA-1 as determined by immunofluorescence staining and have measurable telomerase while control fibroblasts have no measurable telomerase levels as determined by an enzyme-linked immunosorbent assay. In an embodiment, the isolated stem cells are negative for SSEA-3, SSEA-4, Tra-60, and Tra-80 as determined by immunofluorescence staining. In an embodiment, the isolated stem cells respond to TGFβ to form capillary-like structures. In an embodiment, the isolated stem cells do not respond to acidic or basic FGF in a chemotaxis assay but do respond to PDGF-BB in a chemotaxis assay.

The invention provides a method of treating wounds comprising: implanting a matrix in a first wound site of a first living organism; allowing cells to infiltrate the matrix; removing the matrix containing the infiltrated cells from the wound site; removing the infiltrated cells from the matrix to provide isolated stem cells; and applying the isolated stem cells to a second wound site that is on the first or a second living organism. In an embodiment, the second wound site comprises a tendon. In one embodiment, the method further comprises culturing the isolated stem cells before applying the isolated stem cells to the second wound site.

The invention provides a method of deriving, culturing, and differentiating isolated stem cells comprising: implanting a matrix in a wound site of a living organism; allowing cells to infiltrate the matrix; removing the matrix containing the infiltrated cells from the wound site; removing the infiltrated cells from the matrix to provide isolated stem cells; culturing the isolated stem cells in an undifferentiated state; and subsequently differentiating the isolated stem cells into a specific cell type.

### Examples

### Materials and Methods

### Rabbit Wound-Derived Capillary Endothelial Cell (WCEC) Isolation and Culture

WCEC were isolated from wounds created in adult, female New Zealand White rabbits. The rabbits were housed individually, maintained on a 12 hour light-dark cycle, and had access to food and water ad libitum. Wounds were created by implanting polyurethane sponges (3.0 cm×3.0 cm×0.5 cm) subcutaneously into the backs of mice. Sponges were made from large pore, open cell polyurethane foam (AAA Foam, Minneapolis, Minn., USA). Prior to implantation, the sponges were washed in distilled water, boiled two times (30 minutes each) in distilled water, soaked in acetone for 30 minutes, soaked in 95% ethanol for 30 minutes, boiled two additional times (30 minutes each) in distilled water and sterilized by autoclaving. At 12 days post-implantation the sponges were removed aseptically and the cells within the sponge isolated. The tissue adhered to the outside of the sponge was removed and the sponge minced with a scissors. Cell suspensions were obtained by digestion of the sponge with an enzyme cocktail, containing 0.2% protease, 0.5% collagenase and 0.2% DNase (all from Sigma-Aldrich, Inc, St. Louis, Mo.), for two hours at room temperature with gentle stirring. Following the digestion treatment, the cell suspension was washed 3 times in M199 (Gibco, Invitrogen Corporation, Carlsbad, Calif.) containing 10% fetal bovine serum (FBS) (Gibco, Invitrogen Corporation, Carlsbad, Calif.) to inactivate and remove the digestion enzymes. Single cell suspensions were then applied to a PERCOLL (Sigma-Aldrich, Inc, St. Louis, Mo.) gradient and the WCEC isolated from the 30/50% interface. The cells were washed 3 times and cultured on flasks pre-coated with 1% MATRIGEL (BD Biosciences, Bedford, Mass.), in M199 containing 10% rabbit serum/5% FBS in a 37° C. incubator containing 5% CO₂.

### Immunofluorescence Staining

Following isolation, WCEC were stained for the presence of a specific endothelial cell marker. WCEC were placed into MATRIGEL coated 4-well slide chambers and cultured overnight. Staining for the uptake of acetylated-low density lipoprotein (acetylated-LDL) was done using fluorescent Dil-acetyl-LDL (Biomedical Technologies, Inc., Stoughton, Mass.). Cells were incubated for 4 hours at 37° C. in standard culture media containing 10 μg/ml Dil-acetyl-LDL. After culture the cells were examined in a fluorescence microscope using a rhodamine filter set and the cells staining positive for acetyl-LDL uptake enumerated.

### Tube Formation

WCEC were tested for their ability to form capillary-like structures in a three dimensional collagen gel. WCEC were incorporated into a type 1 collagen solution (Vitrogen 100, Celtrix Laboratories, Palo Alto, Calif.) that had been brought to a pH of 7.2 at 4° C. at a concentration of 103 cells/ml. Following dispersion of the cells into the collagen solution, the solution was brought to 37° C. at which point the collagen formed a gel containing the WCEC. The gels containing WCEC were then incubated at 37° C. in the presence or absence of 0.5 ng/ml TGFβ (R&D Systems, Minneapolis, Minn.) for up to 5 days. After culture, the gels were frozen in liquid nitrogen for subsequent preparation of frozen sections. Ten micron frozen sections were made and stained with hematoxylin and eosin or examined directly by Nomarski interference contrast for evaluation of tube formation. Differentiation into capillary-like structures in the presence of TGFβ is an indicator of endothelial character of the cells.

### Proliferation Assays

Proliferation studies of WCEC were undertaken to determine potential mitogens for these cells. Mitogens tested included PDGF-AA (platelet derived growth factor-AA), PDGF-BB, PDGF-AB, basic FGF (fibroblast growth factor), acidic FGF, EGF (epidermal growth factor), and TGFα (transforming growth factor-α) (all from R&D Systems, Minneapolis, Minn., USA). The PDGF's, FGF, EGF, and TGFα are fibroblast growth factors. TGFβ (transforming growth factor-β, from R&D Systems, Minneapolis, Minn., USA) was also tested. TGFβ is not a mitogen but causes endothelial cells to differentiate. WCEC were plated in 1.0% MATRIGEL pre-coated 24-well tissue culture plates at concentration of 5×10³ per ml per well in M199 contained 2.5% FBS and cultured at 37° C. in a 5% CO₂ incubator for 24 hours. At Day 1 the media was removed from the wells and replaced with M199 containing 2.5% FBS with or without the mitogen to be tested and the plates returned to the incubator. At Day 3 each well received fresh media containing the same treatment and returned to the incubator. On Day 4 the media was removed from the wells, the wells rinsed with M199, and M199 containing 0.5% trypsin/EDTA (Gibco, Invitrogen Corporation, Carlsbad, Calif.) was added to detach the cells from the plate. Following detachment, the number of cells was determined by visual counting in a hemocytometer. The ability to stimulate cellular proliferation compared to the M199/2.5% FBS baseline was then determined. WCEC cultured in M199/10% FBS was used as the positive control and was shown to be the maximal level of proliferation attainable.

### Chemotaxis Assays

Chemotaxis studies were performed using a modified 48-well Boyden chamber technique (NeuroProbe, Gaithersburg, Md.). Boyden S, The chemotactic effect of mixtures of antibody and antigen on polymorphonuclear leukocytes, J Exp Med 1962, 115:453-466. The putative chemoattractants, (PDGF-AA, PDGF-BB, PDGF-AB, basic FGF, and acidic FGF) to be tested were placed into the bottom wells of the chamber at concentrations ranging from 0.01-100 ng/ml in M199. A polycarbonate filter with 8 μm pores was placed over the bottom chamber and fixed in place. WCEC (2.5×10⁵ in 40 ul) were placed into the upper chamber in M199. The chambers were then incubated for 4 hours at 37° C. in a humidified 5% CO₂ incubator. Following incubation, the filter was carefully removed and the cells on the top of the filter removed by scraping. The cells, which had migrated to the bottom of the filter, were fixed and stained with Wright's stain. The cells, which had migrated through the filter, were quantified in a microscope and expressed as cells per high power field (HPF). Endothelial cells should respond to FGF and not to PDGF.

### Receptor Studies

To further assess the ability of PDGF-BB and basic FGF to stimulate a biologic response, studies were undertaken to examine the PDGF-BB and basic FGF receptors on WCEC. WCEC were isolated and grown to confluence in 24-well dishes. Various concentrations of ¹²⁵I-PDGF-BB or ¹²⁵I-basic FGF from 0.5-10 ng/ml was added to the tubes and incubated for 1 hour at 4° C. After incubation, the cells were washed with phosphate buffered saline solution (PBS) three times, lysed with 0.5% Triton X-100 and the radioactivity determined in a gamma counter. To determine nonspecific binding, replicates were done containing a 100× concentration of non-labeled PDGF-BB or basic FGF. The specific binding of ¹²⁵I-PDGF-BB and basic FGF at the various concentrations of ligand was analyzed according to the method of Scatchard to determine the Kd of the receptor and the number of receptors per cell. Scatchard G, The attractions of proteins for small molecules and ions, Ann NY Acad Sci, 1949, 51:660-672.

### Tyrosine Kinase Receptor Phosphorylation Studies

As both the PDGF and FGF receptor systems are tyrosine kinase receptors, the method of determining signal transduction, via receptor-mediated phosphorylation, is essentially the same. WCEC were stimulated with either 1.0 ng/ml PDGF-BB or 1.0 ng/ml basic FGF for 30 minutes at 37° C. in MATRIGEL-coated 25 cm² tissue culture flask. Following incubation, the flasks were rinsed three times with ice cold PBS before treatment with 1.0 ml of extraction buffer containing phenylmethylsulfonylfluoride (PMSF), Triton X-100, and sodium orthovanadate (all from Sigma-Aldrich, Inc., St. Louis, Mo.) in PBS. The extract was then applied to 7.5% polyacrylamide/SDS gel and run at 5.0 mA. The gel was then prepared for transfer to a nitrocellulose membrane using a BIO-RAD TRANSBLOT system. Following protein transfer to the nitrocellulose membrane (Immobilon-P, Millipore Corporation, Bedford, Mass.), the blots were incubated with an antibody to phosphoryl-tyrosine (Upstate Biotechnology/Millipore Corporation, Bedford, Mass.) for the detection of phosphorylation product, rinsed, and incubated with an HRP labeled secondary antibody for subsequent staining with an HRP detection system.

### Isolation of Mouse Wound-derived Stem Cells

We chose mice for wound derived stem cell (WDSC) studies because commercially available reagents are readily available for stem cell analysis. The mice were housed in a vivarium, maintained on a 12 hour light-dark cycle, and had access to food and water ad libitum. WDSC were isolated from artificially created wounds in adult, male BALBc/3T3 mice (Jackson Laboratory, Bar Harbor, Me.). Wounds were created by implanting polyurethane sponges (1.0 cm×1.0 cm×0.3 cm) subcutaneously into the backs of mice, one on each side of the backbone. Sponges were made from large pore, open cell polyurethane foam (AAA Foam, Minneapolis, Minn., USA). Prior to implantation, the sponges were washed extensively in numerous changes of alcohol and water and sterilized by autoclaving (as described earlier). At various time points the sponges were aseptically removed and the cells contained within the sponge isolated. The tissue adherent to the outside of the sponge was removed and the sponge minced with a scissors. Cell suspensions were obtained by digestion of the sponge with an enzyme cocktail, containing 0.2% protease, 0.5% collagenase and 0.2% DNase, for two hours at room temperature with gentle stirring. Following the digestion treatment, the cell suspension was washed 3 times in M199 containing 10% FBS to inactivate and remove the digestion enzymes. Single cell suspensions were then applied to a PERCOLL gradient and the WDSC isolated from the 30/50% interface. The cells were washed 3 times and then used for characterization, in vitro and in vivo studies or cultured. Cells for culture were plated onto MATRIGEL-coated flasks and cultured in M199 containing 15% ES-certified FBS (American Type Culture Collection, Manassas, Va.) in a 37° C. incubator containing 5% CO₂.

### Control Cell Culture

Control osteoblast cells (CRL-12557) were cultured in MEM with 10% FBS at 37° C. in a 5% CO₂ incubator. Control L929 fibroblast cells (#CCL-1) were cultured in MEM with 5% horse serum at 37° C. in a 5% CO₂ incubator while 3T3 fibroblasts (CCL-163) were cultured in DMEM with 10% NCS at 37° C. in a 10% CO₂ incubator. Neuronal cells (CRL-2925) were cultured in MEM containing 10% FBS at 37° C. in a 5% CO₂ incubator. All control cells were obtained from American Type Culture Collection, Manassas, Va.

### Immunofluorescence Staining

Cultured WDSC were stained for various stem cell markers including Oct-4, SSEA-1, SSEA-3, SSEA-4, Tra-60, and Tra-80 (Chemicon/Millipore Corporation, Bedford, Mass.). Briefly, WDSC were cultured in M199/15% ES-FBS on slide flasks pre-coated with 1% MATRIGEL and the cells were stained for the presence of various markers. The staining technique was a standard sandwich technique employing a primary antibody for the desired marker and a secondary fluorescein-conjugated antibody against the primary antibody. Briefly, the cells were fixed and permeablized, and nonspecific binding blocked with goat serum. The cells were then stained with a 1:25-1:50 dilution of the primary antibody for 60 minutes. Following primary staining, the cells were washed 3 times and stained for 60 minutes with a 1:50-1:100 dilution of the fluorescein-conjugated secondary antibody for 60 minutes. After washing 3 times, the cells were examined in a fluorescence microscope with a fluorescein filter set.

### Telomerase Assay

Telomerase levels are known to be high in undifferentiated stem cells. Telomerase levels were determined non-quantitatively using a TRAPeze ELISA Telomerase Detection Kit (Chemicon/Millipore Corporation, Bedford, Mass.). ELISA stands for an enzyme-linked immunosorbent assay. Briefly, WDSC were isolated and homogenized in lysis buffer. The telomerase present in the sample adds telomeric repeats (GGTTAG) onto the 3′ end of a biotinylated telomerase substrate. This is followed by a PCR amplification with dCTP labeled with dinitrophenol (DNP). Following amplification, the telomeric repeat amplification products are now tagged with biotin and DNP. The products are immobilized onto streptavidin-coated microtiter plates, detected by anti-DNP antibody conjugated with horseradish peroxidase and relative telomerase activity determined in a plate reader.

### Osteoblast Differentiation Studies

WDSC were examined for their ability to differentiate into osteoblasts following a treatment regimen known to stimulate osteoblast cell formation. WDSC were treated with M199 containing 15% ES-FBS and supplemented with 10 nM β-glycerophosphate, 170 μM ascorbic acid, 100 nM dexamethazone (Sigma-Aldrich, Inc., St. Louis, Mo.), and 100 ng/ml bone morphogenic protein 4 (R&D Systems, Minneapolis, Minn.). The media was replaced on day 6 and every 3 days thereafter. At various times after treatment, the cells were examined for osteoblast markers as described below.

### Alizarin Red Staining

Following treatment with transformation cocktail, WDSC cultures in 25 cm³ flasks were rinsed with PBS once, fixed with 10% formaldehyde, rinsed with distilled water, then stained with 3 mL of 40 mM Alizarin Red S for 20 minutes. This was then washed with distilled water to remove excess stain for observation.

### Analysis of WDSC Gene Changes Using PCR

Transformation of WDSC into osteoblasts was monitored by gene analysis using PCR. The following genes, which have been shown to be regulated during osteoblast differentiation and development, were followed over the course of differentiation: alkaline phosphatase (Genbank NM_{—}007431), collagen, type 1, alpha 2 (GenBank NM_{—}007742), bone gamma-carboxyglutamate protein 2 (GenBank NM_{—}001032298), integrin binding sialoprotein (GenBank NM_{—}008318), runt related transcription factor (GenBank NM_{—}009820), collagen, type 1, alpha 1 (GenBank NM_{—}007743), neighbor of punc E11 (GenBank NM_{—}020043), bone gamma-carboxyglutamate protein 1 (GenBank NM_{—}007541), secreted phosphoprotein 1 (GenBank NM_{—}009263), phosphate regulating gene (GenBank NM_{—}011077), hemogen (GenBank NM_{—}053149), and glyceraldehyde-3-phosphate dehydrogenase (GenBank NM_{—}008084). WDSC were examined on days 6 and 9 following initial treatment with the differentiation media.

### Neuronal Differentiation Studies

WDSC were examined for their ability to transform into neuronal cells following a treatment regimen known to stimulate neuronal cell formation. The treatment was a replacement of growth media with MEM supplemented with 10 uL/mL of Neuronal Differentiation Media (R&D Systems, Minneapolis, Minn.), 10⁻⁶M retinoic acid, and 1.5 ug/ml purmorphamine (Stemgent, Cambridge, Mass.). The media was exchanged every 3 days in culture. A neuronal stem cell line (ATCC # CRL-2925) was treated in the same manner for use as a positive control for nestin upregulation.

### Immunofluoresence Staining

Cells to be stained were washed twice with PBS, fixed with paraformaldehyde, then washed with 1% Bovine Serum Albumin (BSA, Sigma-Aldrich, Inc., St. Louis, Mo.) in PBS, permabilized and blocked with 10% normal donkey serum (Millipore, Bedford, Mass.), 1% BSA and 0.3% Triton X-100 (Sigma-Aldrich Inc., St. Louis, Mo.) in PBS. The primary antibodies for nestin were incubated overnight at 4° C. then washed three times with 1% BSA/PBS. The secondary antibodies were diluted 1/50 and incubated for 60 minutes then washed three times with 1% BSA/PBS and prepared for visualization.

### RNA Isolation and Reverse Transcription

Total cellular RNA was isolated from cultured mouse cells (1×10⁶) that were frozen and stored at −80° C. in RNA protect solution (Qiagen, Inc., Valencia, Calif.) at the time of harvest. A mini RNAeasy Kit (Qiagen, Inc., Valencia, Calif.) was used to extract RNA from cells per the manufacturer instructions using the QiaShredder spin column (Qiagen, Inc., Valencia, Calif.) for homogenization. Concentration, purity, and integrity of all RNA samples were determined by measuring the A260, A260/280 and A260/230 readings and ratios using a Nanodrop 2000c (Thermo Scientific, Wilmington, Del.). Synthesis of cDNA for quantitative RT-qPCR was performed using RT2 First Strand Kit (SA Biosciences, Frederick, Md.) according to manufacturer instructions with a starting template of 500 ng of total RNA per sample. All cDNA reactions were diluted with 91 ul of H₂0 and placed on ice or stored at −20 C. until RT-PCR was performed. Appropriate numbers of no template and reverse transcription template control samples were run to determine presence of genomic DNA contamination in our assay.

### Real-Time PCR

A set of custom RT-PCR arrays were designed and created to measure the expression levels for a specific set of genes targets related to osteoblast development and differentiation. The sequences for the genes of interest were obtained from GenBank and that list was sent to SA Biosciences (Frederick, Md.) who designed, made, and coated those primers onto PCR plates using their proprietary method.

All qualitative RT-PCR was done using iCycler IQ5 thermocycler (Bio-Rad Laboratories, Richmond, Calif.) equipped a MyIQ optic single color detection system. All diluted cDNA samples were added to a precise mixture of 2× SYBR Green/Fluorescein qPCR Master Mix (SA Biosciences, Frederick, Md.) and water according to the RT2 Custom Profiler array specifications to give final volume of 25 ul per well. The reaction mixtures were then amplified after 10 minute activation and denaturation step at 95° C. followed by a three step cycling program of 40 cycles of 15 seconds at 95° C., 10 seconds at 55° C. and 15 seconds at 72° C.

Melt curves were done on all runs to determine the presence of primer dimers and other artifacts that could invalidate array results. Data analysis of RT-PCR results was done by importing a spreadsheet of raw Ct values into a SA Bioscience web software tool designed for their custom arrays. Any Ct values which greater that 35 were considered and read as a negative cell. β-actin was used as the control reference gene for normalization to determine the fold change regulation changes among samples using the Livak method. Livak et al., Analysis of relative gene expression data using real-time quantitative PCR and the 2(-Delta Delta C(T)) method, Methods 2001, 25:402-408.

### Results

### Evaluation of Rabbit Wound-Derived Cells as Putative Capillary Endothelial Cells

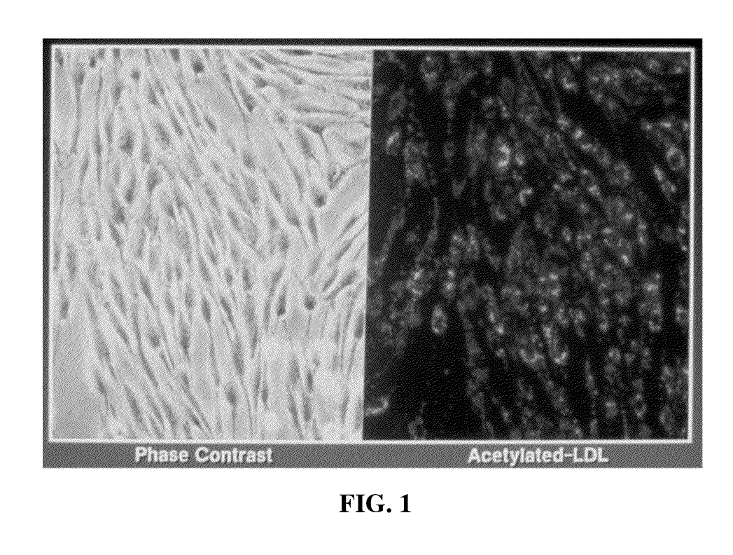

Utilizing specific stains and functional analysis, characterization studies were performed to determine the endothelial nature of this population. As shown in FIG. 1, immunofluorescence staining for acetylated-LDL uptake was positive and demonstrated the endothelial nature of this cell population.

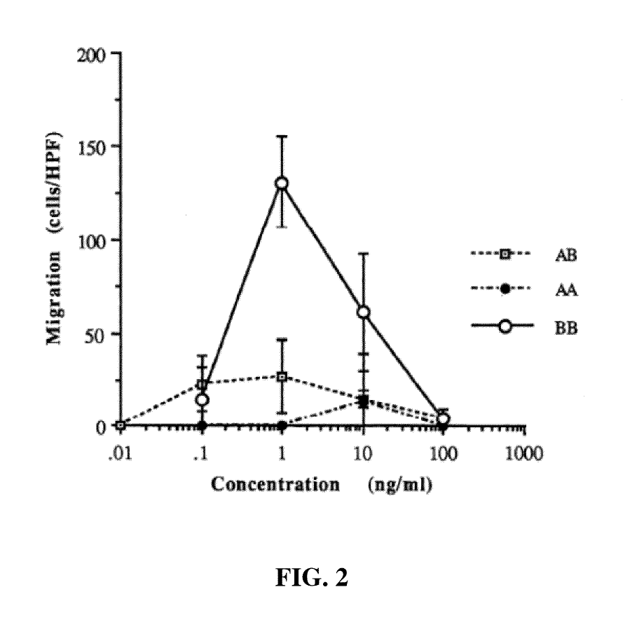

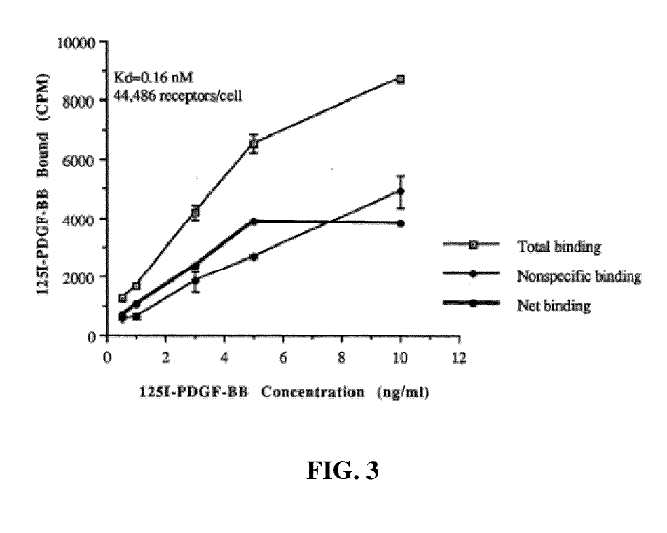

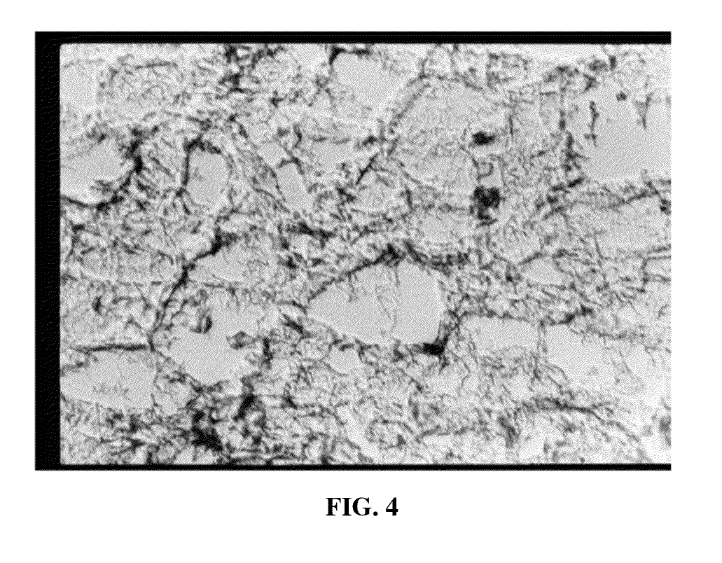

The functional studies performed centered on the processes the endothelial cell undertakes during angiogenesis: chemotactic migration, proliferation, and differentiation. Studies of chemotaxis were performed in modified Boyden chambers using the known endothelial chemoattractants basic FGF and acidic FGF. The three isoforms of PDGF, a known chemoattractant for inflammatory and mesenchymal cells, were tested as well. Both acidic and basic FGF failed to elicit a chemotactic response in these cells when tested over a wide range of doses (0.01-100 ng/ml). However, when the isoforms of PDGF were examined there was a demonstrable response, primarily to PDGF-BB. As shown in FIG. 2, PDGF-BB elicited a strong, dose dependent chemotactic response, which was maximal at 1-3 ng/ml. PDGF-AA was negative while PDGF-AB demonstrated a very slight response. These results indicate the presence of exclusively beta-subunit PDGF receptors on these cells. These results led us to examine WCEC for the presence of PDGF receptors. As demonstrated in FIG. 3, studies utilizing ¹²⁵I-PDGF-BB demonstrated specific receptors for PDGF-BB with approximately 45,000 receptors/cell and a Kd of 0.1-0.2 nM. The binding of ¹²⁵I-basic FGF to WCEC was also determined. While WCEC did bind ¹²⁵I-basic FGF, the binding was nonsaturable and did not appear to be receptor mediated as it was easily displaceable by 2M NaCl, indicating a heparin-like receptor mediated binding.

The ability to enhance proliferation of these cells was tested using the same growth factors (FGF and PDGF) as well as EGF, TGFα, and TGFβ. WCEC were cultured in 2.5% FBS/M199, a concentration of serum providing for half maximal growth as compared to growth in 10% FBS/M199. Various concentrations of the putative mitogens were added at days 1 and 3. The cells were harvested on day 4 and enumerated using a hemocytometer. None of the mitogens tested were able to elicit a significant proliferative response over doses ranging from 0.01-100 ng/ml. Because of these results, the mitogens were also tested in the presence of 1% or 10% FBS utilizing the same assay system. Again, all of the mitogens tested failed to elicit a proliferative response in WCEC.

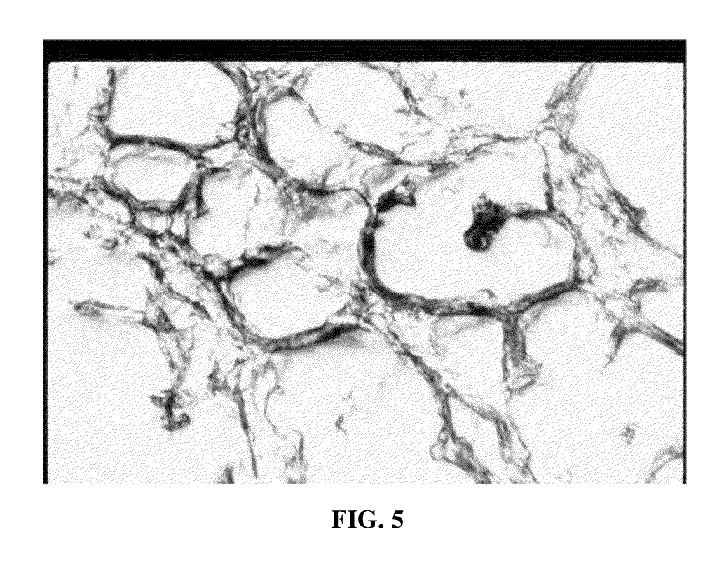

Finally, these cells were tested for their ability to differentiate into capillary-like structures. Cells were isolated and incorporated into 3-dimensional collagen gels in the presence or absence of TGFβ at a concentration of 0.5 ng/ml. The gels were then cultured for 3-5 days. After culture, the gels were frozen in liquid nitrogen for subsequent preparation of frozen sections. Frozen sections were made and stained with hematoxylin and eosin for evaluation by bright-field microscopy or examined directly by Nomarski interference microscopy. As shown in FIG. 5, cells in the presence of TGFβ formed tube-like structures representing the ability to form capillaries.

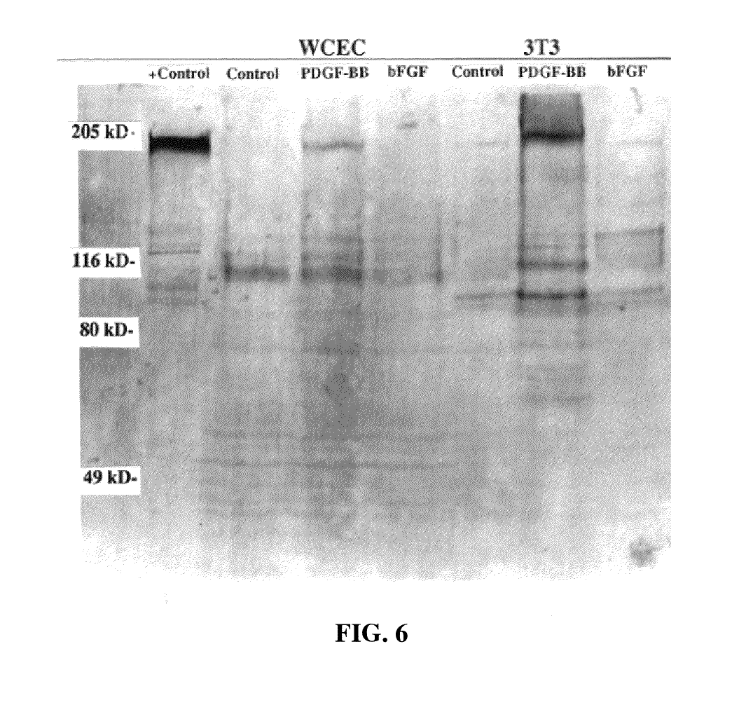

The results of these studies provided a glimpse into the functional responsiveness of the WCEC, which was unlike any previously described endothelial cell population. These cells were responsive to PDGF-BB, but not basic and acidic FGF. To further understand this phenomenon studies were undertaken to evaluate the tyrosine kinase signal transduction pathway of both the PDGF and FGF receptors. WCEC were treated with either 1.0 ng/ml PDGF-BB or 1.0 ng/ml basic FGF and the ability of these two ligands to stimulate receptor-mediated tyrosine kinase activity was determined. FIG. 6 demonstrates the ability of PDGF-BB and basic FGF to induce tyrosine kinase activity in control 3T3 fibroblasts. When WCEC were treated with PDGF-BB, the same receptor mediated stimulation occurred as in the control fibroblasts while treatment of these cells with basic FGF produced no stimulation of tyrosine kinase activity.

### Evaluation of Mouse Wound-Derived Capillary Endothelial Cells as Putative Stem Cells.

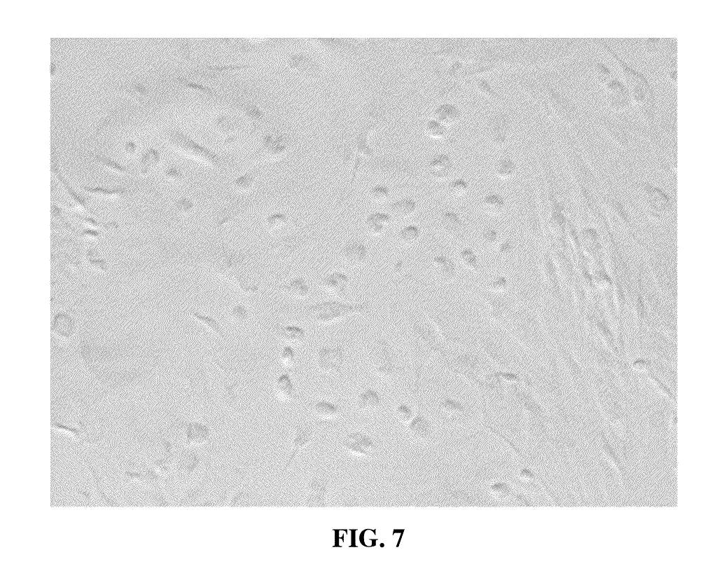

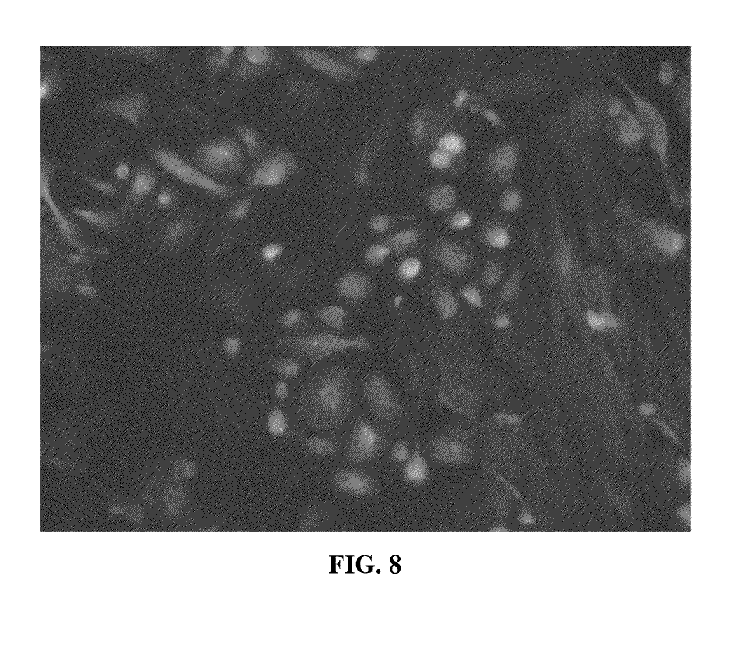

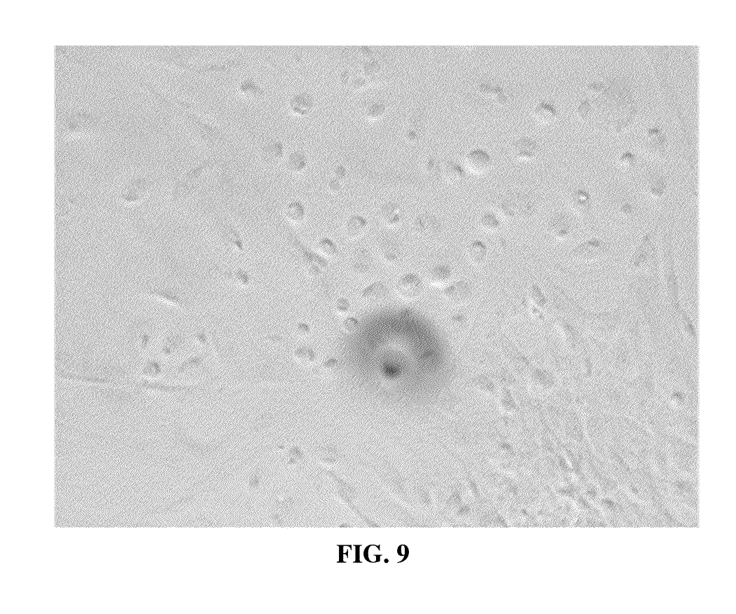

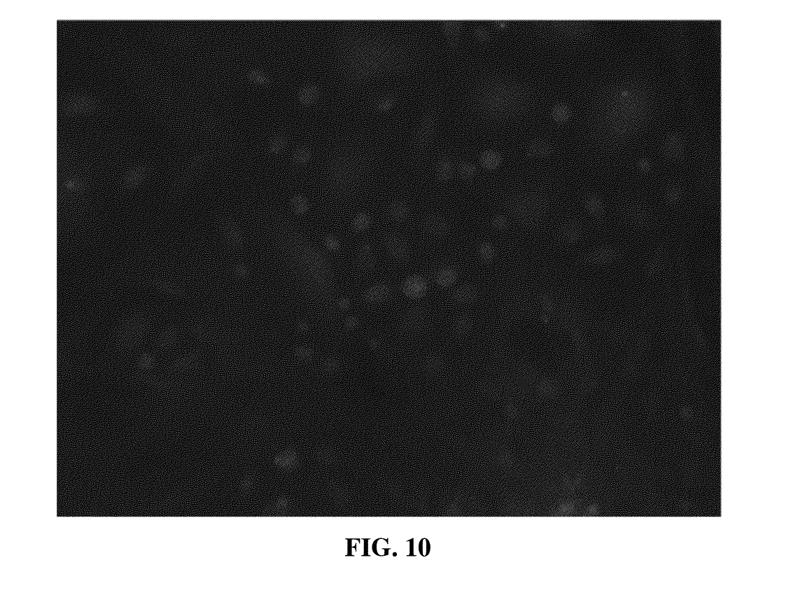

Sponge implants were removed from the mice at various days following implantation. Cell populations were then isolated from individual sponges as described in the Materials and Methods section. Following isolation, the cells were washed extensively and enumerated using a hemocytometer. Trypan Blue was utilized to determine cell viability and only cells excluding the dye were enumerated. Cell viability was always greater than 95%. Following isolation, WDSC were cultured in slide flasks under standard culture conditions. After allowing the WDSC to adhere, the cells were stained for the presence of Oct-4 utilizing a primary antibody specific to Oct-4. As shown in FIGS. 7 and 8, WDSC demonstrated positive staining for the presence of the Oct-4 antigen while control cultures of terminally differentiated fibroblasts were completely negative. Subsequently, WDSC were stained for the presence of additional stem cell markers including SSEA-1, SSEA-3, SSEA-4, and Tra-60. Of these additional markers, only SSEA-1 showed any positive staining. As shown in FIGS. 9 and 10, WDSC stained for the presence of SSEA-1 demonstrated significant positive staining when compared to terminally differentiated fibroblasts.

Further characterization of these was performed to determine the telomerase activity. The results of these studies are shown in Table 1 below. WDSC were analyzed for telomerase activity using a commercially available kit as described in the Materials and Methods. WDSC had demonstrably higher levels of telomerase activity when compared to 3T3 fibroblasts. This result again provides evidence that these cells possess stem cell-like properties.

TABLE 1
Telomerase activity of WDSC

| | | Heat-inactivated | Net Change in |
|---|---|---|---|
| | Test Sample | Test Sample | Absorbance |
| Positive Control | 1.511 | 0.314 | 1.197 |
| 3T3 Fibroblasts | 0.071 | 0.093 | −0.022 |
| WDSC | 0.155 | 0.049 | 0.108 |

WDSC were isolated from sponges 7 days post-implantation as described in the Materials and Methods. The positive control was supplied in the kit. Heat treatment inactivates the telomerase; positive telomerase activity is indicated by a net increase in absorbance.
In vitro differentiation of WDSC to osteoblasts

WDSC were differentiated into osteoblasts by treatment with BMP-4, β-glycerophosphate, ascorbic acid, and dexamethazone, a cocktail that has been shown to cause embryonic stem cell (ESC) differentiation into osteoblasts. Following treatment with this differentiation media, the cells were examined for their ability to stain with alizarin red S, a stain specific for calcium mineralization in osteoblasts.

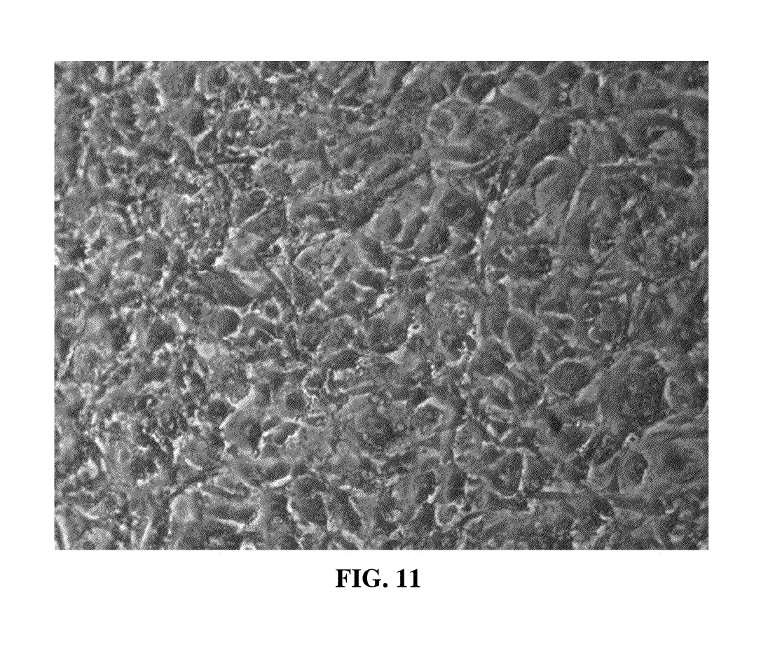

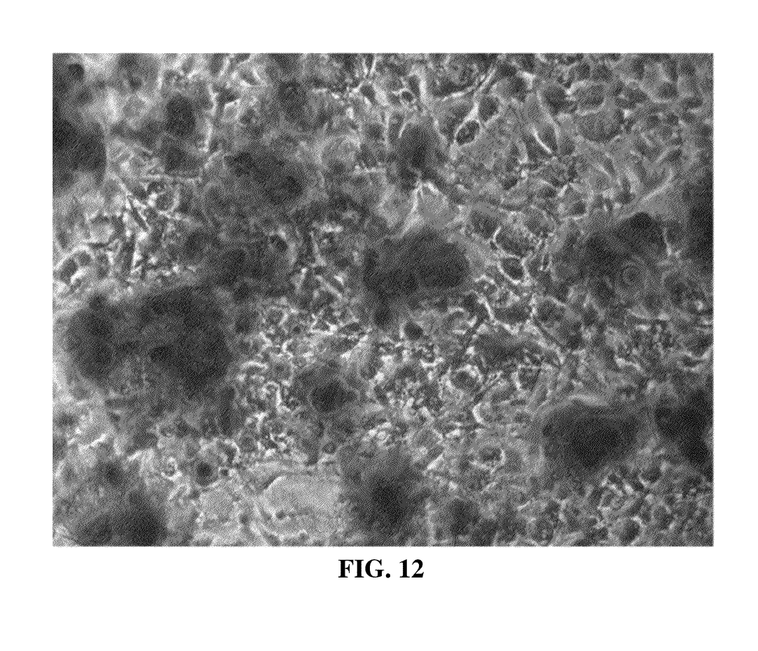

WDSC which had been treated with osteoblast differentiation media demonstrated significant staining for calcium mineralization when stained with alizarin red S (FIGS. 11 and 12).

The data shown in Table 2 below demonstrate the ability of WDSC to upregulate osteoblast genes and differentiate into osteoblast-like cells following stimulation. WDSC were examined 6 and 9 days after treatment with osteoblast differentiation media. The genes of interest were analyzed by comparison to an osteoblast cell line (ATCC CCL-12557) as described in the Materials and Methods. As Table 2 shows, a number of the osteoblast genes are up-regulated in WDSC upon culture alone at days 6 and 9. However, treatment with the osteoblast differentiation media significantly up-regulates a number of the osteoblast genes at days 6 and 9 of treatment, including alkaline phosphatase, bone gamma-carboxyglutamate protein 2, integrin binding sialoprotein, and runt related transcription factor, and also up-regulates bone gamma-carboxyglutamate protein 1 at day 9 of treatment.

TABLE 2
Effect of osteoblast differentiation media on
WDSC osteoblast gene expression

| | WDSC | WDSC | WDSC | WDSC | WDSC |
|---|---|---|---|---|---|
| Gene | Day 0 | Day 6− | Day 6+ | Day 9− | Day 9+ |
| Alkaline phosphatase | −112.99 | −13.45 | 19.43 | −3.89 | 16.68 |
| Collagen, type 1, alpha 1 | −1.09 | 35.02 | 19.29 | 38.85 | 15.24 |
| Bone gamma- | −20.82 | −7.78 | 5.43 | −1.88 | 6.59 |
| carboxyglutamate | | | | | |
| protein 2 | | | | | |
| Integrin binding | −243.88 | −99.73 | 6.15 | −50.56 | 4.96 |
| sialoprotein | | | | | |
| Runt related transcription | −2.14 | 2.79 | 4.47 | 2.39 | 3.05 |
| factor 2 | | | | | |
| Collagen, type 1, alpha 2 | 3.58 | 58.08 | 44.94 | 70.03 | 36.00 |
| Neighbor of Punc E11 | −1.68 | 8.28 | 5.43 | 9.71 | 4.89 |
| Bone gamma- | −8.69 | −2.58 | 2.16 | −2.33 | 3.41 |
| carboxyglutamate | | | | | |
| protein 1 | | | | | |
| Secreted | 7.21 | 5.28 | 8.40 | 26.72 | 10.63 |
| phosphoprotein 1 | | | | | |
| Phosphate | −1.43 | −1.43 | −1.43 | −1.05 | −1.43 |
| regulating gene | | | | | |
| Hemogen | 1.00 | 1.00 | 1.00 | 1.00 | 1.00 |
| Glyceraldenhyde- | 1.00 | 1.00 | 1.00 | 1.00 | 1.00 |
| 3-phosphate | | | | | |
| dehydrogenase | | | | | |

WDSC were isolated from sponges 7 days post-implantation as described in the Materials and Methods. The values shown represent the fold increase or decrease compared to an osteoblast cell line (ATCC CCL-12557). WDSC Day 0: gene expression of WDSC at Day 0, prior to culture. WDSC Day 6− and WDSC Day 9−: gene expression of untreated WDSC at days 6 and 9 respectively. WDSC Day 6+ and WDSC Day 9+: gene expression of WDSC treated with differentiation media at days 6 and 9 respectively.

### In Vitro Differentiation of WDSC to Neuronal-Like Cells

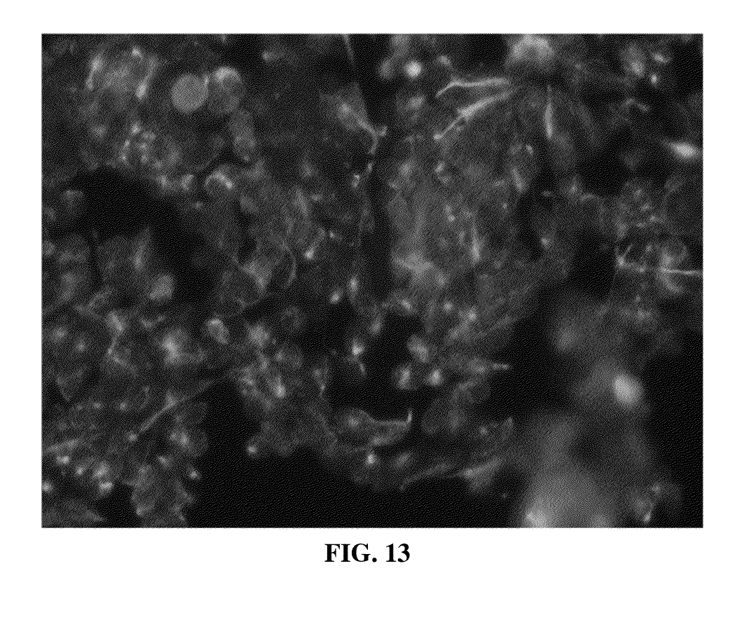

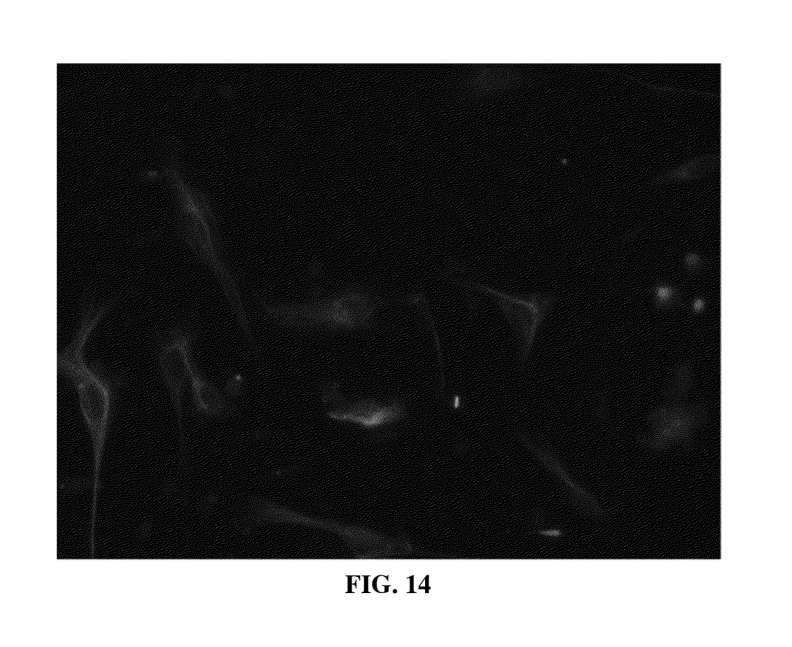

Transformation of WDSC into neuronal-like cells was monitored by staining. for nestin, a protein known to be up-regulated during neuronal transformation. WDSC were differentiated into neuronal-like cells by treatment as described in the Materials and Methods. Growth media was supplemented with 10 uL/mL of Neuronal Differentiation Media, 10⁻⁶M retinoic acid and 1.5 ug/mL purmorphamine. Following treatment with this differentiation media, the cells were examined for their ability to stain for nestin, a marker specific for neuronal cells. Staining of differentiated WDSC showed significantly greater staining with nextin antibody when compared to undifferentiated WDSC and was comparable to the staining of neuronal stem cells treated with the same differentiation protocol (FIGS. 13 and 14). Analysis of the appearance of nestin was monitored over a 12 day span and treated WDSC demonstrated upregulation of nestin staining over 6-12 days after initiation of treatment.

### Discussion

Angiogenesis is a fundamental component of granulation tissue formation and wound repair. As shown in our in vivo studies using microvascular casting, wound angiogenesis starts with margination of inflammatory cells on the post capillary venules. The post capillary venules then enlarge and at 72 hours after wounding, capillary buds are formed. These elongate and eventually form a capillary loop. A new bud forms at the apex of the capillary loop. This process continues to form a new capillary network at the leading edge of the granulation tissue while the feeding capillaries mature. In these studies we never observed margination or capillary bud formation from the arterial side of the network.

Using the wound sponge model and surgically removing all adherent tissue from the edge of the sponge physically removes any tissue that is not newly grown into the sponge. We used this physical barrier to only harvest cells from newly forming granulation tissue. This process provides large numbers of cells with minimal manipulation. By not requiring cell sorting, extensive clonal expansion or repeated passages in culture, this technique provides cells that are closer to those in vivo.

We hypothesized that these cells are endothelial because they stain for acetylated-LDL uptake and make capillary tubes when exposed to TGFβ in vitro. Unlike other arterial and venous endothelial cells, they do not proliferate when exposed to FGF but are able to proliferate in the presence of serum. They have receptors for PDGF-BB and this growth factor is able to elicit a chemotactic response in these cells but not a proliferative response.

The observation that these cells could be stem cells was triggered by their culture morphology and the reported observation that endothelial cells associated with tumor angiogenesis had stem cell markers. These studies hypothesized that circulating bone marrow stem cells were incorporated into the growing capillary network around tumors. This is certainly possible, but our in vivo studies showed no evidence of incorporation of any cells other than those from the post capillary venous network. These observations stimulated us to see whether the WCEC were indeed stem cells that were stimulated to differentiate into capillaries in the wound space by TGFβ.

Initial in vitro studies used cell markers for stem cells. These cells were positive for OCT 4 and SSEA-1 and did not stain for SSEA-2, SSEA-3 or TR60. Staining for Oct-4 was strongest in early passage cell cultures with 95% of the cells with positive markers. As cells were successively cultured, they progressively lost their Oct-4 positive phenotype. This staining pattern indicated an adult stem cell population with a unique phenotype.

Telomerase activity was also elevated in primary cell cultures. The combination of Oct-4, SSEA-1 and telomerase positivity strongly suggests that these cells had significant similarity to embryonic stem cells. These data strongly suggest that these post venular capillary endothelial cells, when signaled to migrate into a wound are functionally pluripotent stem cells.

If that is true then we should be able to stimulate these cells to differentiate into other functional cells. We chose to create osteoblasts and neurons. Using published techniques we were able to produce cells from WDSC with up-regulated genes similar to known osteoblast cells. In addition, these cells demonstrated calcium mineralization nodules when stained with alizarin red S. Treatment of WDSC and neuronal stem cells with a with media supplements known to stimulate neuronal stem cells into neurons also stimulated an upregulation of nestin in WDSC.

These studies could significantly change our understanding of wound healing angiogenesis and provide a role for embryonic like stem cells in angiogenesis and tissue repair. These studies may provide a basis for understanding a potential pathway that causes differentiation of stem cells in the wound into capillaries and eventually regulating a scarring versus a regenerative response. WDSC provide a source of adult, autologous, pluripotent, stem cells for research and therapeutic applications.

### Wound Healing Angiogenesis

These studies along with our previously published work on the morphology of wound healing angiogenesis provide insight into the cellular and signaling pathways associated with angiogenesis in wounds and possibly other physiologic and pathologic processes.

Post-capillary venular endothelial cells respond to signals from the wound space that cause contraction of the cells and margination of circulating neutrophils and macrophages. By 48 hours, new capillary buds are formed that continue to elongate to form capillary loops. These cellular events are probably due to the production of vascular endothelial cell growth factor (VEGF) and PDGF-BB by platelets α-granules. VEGF produces endothelial cell contraction and proliferation while PDGF-BB causes chemotaxis of the post-capillary venule endothelial cells up a chemical gradient. As TGFβ concentrations rise, these cells form tubes that become functional capillaries.

Role for Adult Stem Cells in Angiogenesis Previous studies showed that endothelial cells from tumor angiogenesis stained positive for stem cell markers. They hypothesized that these cells were circulating bone marrow stem cells that were incorporated into the capillary network. Another possible explanation is that the post capillary endothelial cells are pluripotent stem cells that migrate and divide in the early stages of angiogenesis and then differentiate under the influence of TGFβ to form capillaries.

### Scarring Versus Regeneration

The presence of pluripotent stem cells in adult mice wounds is supported by our findings that these cells are naturally positive for Oct-4 using immunofluorescent staining and real time PCR, that they are telomerase positive, and stain for SSEA-1. In addition, we were able to stimulate differentiation of these cells into osteoblasts and neuronal-like cells, as well as capillaries.

The presence of these cells in adult healing wounds could explain why mammalian wounds scar and do not regenerate new tissue. As these stem cells populate the wound space they are immediately signaled by TGFβ to differentiate into capillary endothelial cells to form the new capillary vasculature for the developing granulation tissue. In other species that regenerate tissue, these stem cells are signaled to form all of the tissue types that eventually reform the wound into functional tissue.

Further understanding of this process could lead to therapies that control this differentiation process and lead to regeneration instead of scarring in mammalian wounding.

### Source of Adult Pluripotent Stem Cells for Therapy and Research

The process we have developed to isolate, purify and culture these cells results in the procurement of large numbers of stem cells without extensive manipulation and the use of cell sorting. In these studies we used small implanted sponges in mice, which caused the number of cells to be limited by the surface area of the sponge implants. In larger animals and humans the size of the sponge can be significantly larger, theoretically resulting in much larger cellular harvests.

This could facilitate the progress of stem cell therapies by providing a source of large numbers of autologous, pluripotent stem cells with minimal tissue and cellular manipulation.

### Use of Stem Cells for Tendon Repair

### Study 1

Tendon ruptures are common sports-related injuries that can be treated surgically by use of sutures followed by the use of immobilization to allow the tendon to heal. However, tendon repair by standard techniques is associated with a long healing time and often suboptimal repair. Methods to enhance tendon repair time as well as quality of repair are currently an unmet clinical need. Our hypothesis was that introduction of a unique stem cell population at the site of tendon transection would result in improved rate of repair.

To harvest and collect stem cells a polyurethane sponge (1.0 cm×1.0 cm×0.3 cm) was implanted subcutaneously in donor rats and allowed to collect the circulating stem population within the sponge. Sponges were made from large pore, open cell polyurethane foam (AAA Foam, Minneapolis, Minn., USA). After two weeks the sponges were retrieved and the cells isolated. Tendons of 24 Sprague Dawley Rats were transected and suture repaired with Mason Allen stitches (3 cm length). In half of the rats, a poly(glycolic) acid (PGA) nonwoven scaffold seeded with allogeneic stem cells was attached only to the defect site. The other half was treated with suture alone to serve as controls. One group was randomized to biomechanical testing.

Animals were randomized to a 2 or 4 week time group. At the time of necropsy tendons were harvested, fixed in formalin, decalcified and paraffin embedded. All researchers were blinded to the treatment groups and evaluated histological slides using the Soslowsky scoring system. This scoring system takes into account the orientation and degree of organization of the repaired tendon tissue. Score range from 0-3 with 0 being normal tendon architecture and 3 characterized by marked changes with greater than 50% disorganized.

All animals enrolled in the study tolerated the surgery well with no post-operative complications. Histology results are reported below in Table 3.

TABLE 3

| Time Post-op | Group | Histology score |
|---|---|---|
| 2 weeks | Control | 2.6 |
| | Experimental | 0.6 |
| 4 weeks | Control | 2.2 |
| | Experimental | 0.8 |

The two week group demonstrated a significant improvement in repair compared to controls with no failures. There were minimal inflammatory cells associated with the cell transplant group. There was a complete bridging of the transection site with parallel collagen fiber arrangement. Location of the PGA scaffold was evident in all sections examined. There was a significant improvement in biomechanical strength attained at the two week time point compared to controls. Control suture alone repairs resulted in repair characterized by collagen disorganization and instances of lack of bridging of tendon tissue.

The use of this population of stem cells as an adjunct in tendon repair demonstrates an improved level of organization not previously observed using protein therapy or tissue engineering technologies previously evaluated. The clinical translation of harvesting a patients cells is a minimally invasive and provides easy harvest for tendon repair procedures.

### Study 2

Circulating Stem cells (CSCs) were isolated by implantation of a polyurethane sponge (1.0 cm×1.0 cm×0.3 cm) subcutaneously for two weeks in a group of ten male Sprague Dawley Rats. Sponges were made from large pore, open cell polyurethane foam (AAA Foam, Minneapolis, Minn., USA). The sponges were then retrieved and the cells located within the sponge removed by mild enzymatic digestion. The achilles tendons in 51 adult Sprague Dawley Rats were transected to simulate tendon rupture. Immediately after transection, the tendons were suture repaired with or without a scaffold+CSC. In half of the animals, a biodegradable scaffold seeded with the allogenic CSCs was placed as an onlay to the defect site in addition to the suture repair. The other half was treated with suture alone to serve as controls. After 3 days, 7 days, 2, 4 or 6 weeks post surgery, animals were sacrificed tendons were dissected for histological or biomechanical analysis.

Histological samples from scaffold and control groups were fixed, embedded, sectioned and stained with hematoxylin and eosin. Histological evaluation using the Soslowsky score was conducted on samples from the 2 and 4 week time points. Using this scoring system, where a score of 0 corresponds to normal tendon, and score of 3 indicates marked changes in the collagen organization. Soslowsky et al., 1996, J of Shoulder & Elbow Surgery 5:383. Biomechanical testing was performed at 2, 4, and 6 weeks post surgery with samples treated with scaffold+CSCs. Tendons were tested in uniaxial tension on an Instron testing frame while submerged in a PBS bath. Specimens were mounted in hydraulic grips between two roughened surface plates and sand paper. A pre-load of 0.5N was applied to the specimens, and the length of the specimen was recorded. Tensile displacement was applied at a strain rate of 0.1%/sec until failure. The tensile modulus (E) was determined from the linear portion of the stress-strain curve, and the ultimate tensile strength (UTS) from the maximum load and fractured surface area. Elastic toughness (K) was also calculated numerically using a Reimann sum method. Results were compared against previously published control samples, using the same suture tendon repair described in this study, Dines et al., 2007, Trans ORS, Paper #27.

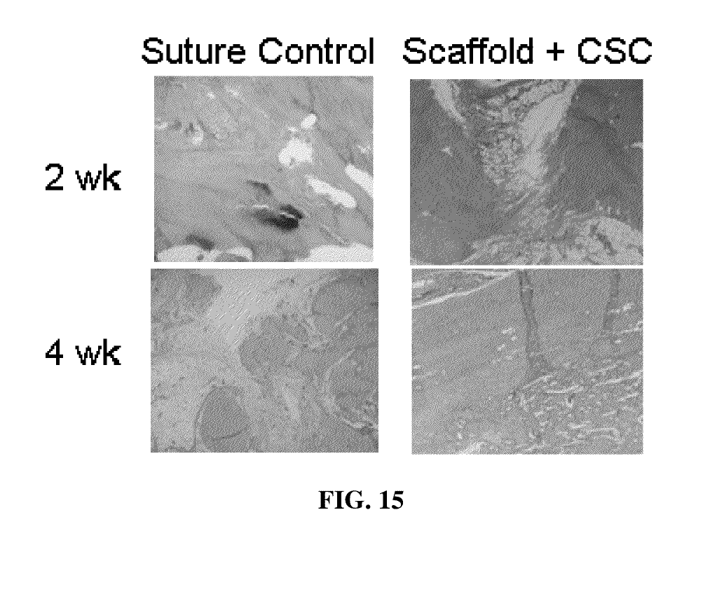

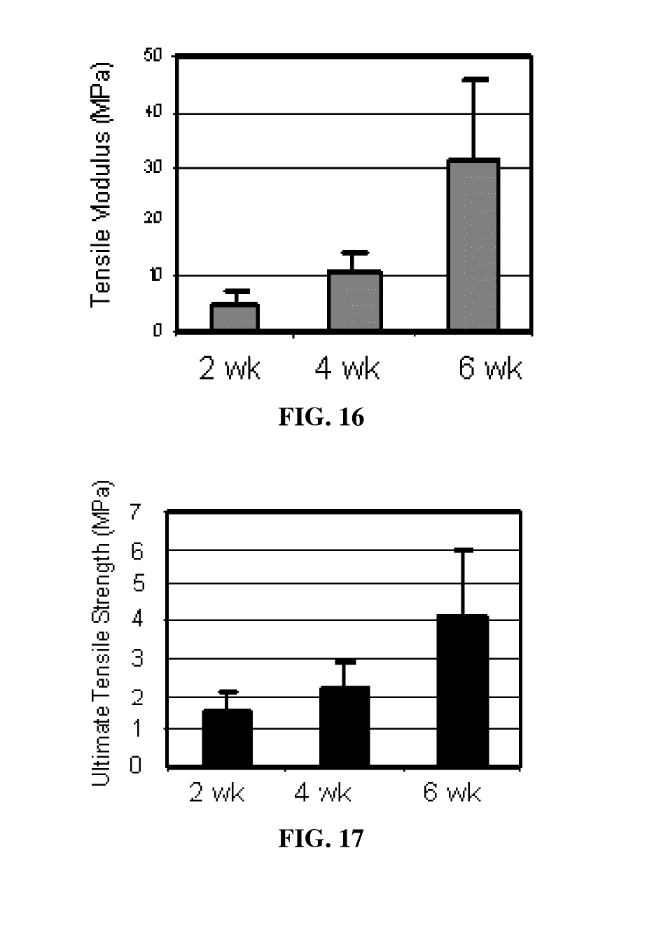

Analysis of the histological scores of the scaffold+CSC group demonstrated significant improvement in repair compared to suture-only controls. Both the three and seven day groups demonstrated minimal repair in both experimental and control animals By 2 weeks, the scaffold+CSC group had an average histological scores of 0.6±0.4 SD, which was significantly greater than the repair seen in the control only group (2.6±0.7SD; p<0.05). The scaffold repair demonstrated complete bridging of the transection site with parallel collagen fiber arrangement (FIG. 15). Control repairs resulted in a repair characterized by collagen disorganization and instances of lack of bridging of tendon tissue (FIG. 15). By 4 weeks, both groups showed a continuing trend of healing, with the scaffold group exceeding the histological quality of the tissue repair (FIG. 15). The scaffold+CSC group had a decreasing cross sectional area with time, (0.167±0.04 at 2wk vs. 0.134±0.03 at 6wks, p<0.05). This was also associated with a significant increase in the UTS of the tendons, reaching 4.2 MPa by 6wks (FIG. 17; p<0.05). An increase in the tensile modulus was seen over time (FIG. 16), reaching 31 Mpa by 6wks (p<0.05). The scaffold+CSC group also achieved a significantly higher elastic toughness by 6 weeks.

In this study, circulating stem cells were found to significantly improve the rate and quality of tendon repair compared to standard suture only repairs. A more complete and physiological repair was seen both histologically and biomechanically. Although the use of MSCs have been associated with ectopic bone and cartilage formation (see Harris et al., 2004, J. Orthop. Res. 998-1003:22), there was no evidence of this occurrence in our samples. Of note, there was no increase in angiogenesis within the tendon tissue, which if present could translate into reduced biomechanical strength. This improvement in tendon tissue regeneration in the absence of increased angiogenesis suggests either unique cellular qualities exhibited by our CSCs or a novel mechanism facilitating tendon repair. It is unclear as to whether the repair was primarily due to the implanted cells on scaffold, or whether the presence of the CSCs stimulated the repair process of the native tendon fibroblasts.

The addition of circulating stem cells to the site of injury also appears to have improved the strength of the repair tissue as demonstrated via biomechanical testing. The UTS, tensile modulus, and the elastic toughness were found to be increase over time, reaching significantly higher values than suture only controls, as previously described in Dines J et al., 2007, Trans ORS, Paper #27. This is clinically noteworthy as most tendon injuries have been shown to heal with a final strength that is markedly below pre-injury baseline strength. Bruns et al., 2000, Knee Surg Sports Tramatol Arthrosc. 364-9:8. A decrease in CSA at the site of injury was also found to be associated with the addition of circulating stem cells suggesting a higher degree of remodeling. Limiting scar formation associated with a tendon injury is crucial especially in flexor tendon lacerations of the hand where repairs must not only be strong, but also capable of passing through the tendon sheath and the associated pulley system of the fingers.

In future studies, the addition of controlled mechanical loading during the repair process will be explored. This could be useful to evaluate both the histological and biomechanical consequences of early range of motion from a practical standpoint and may contribute to new post repair rehabilitation protocols in humans. Cell labeling for in vivo tracking of the cells will also be performed to determine the CSCs role in this enhanced repair process. Cell-to-collagen ratio has been evaluated in stem cell seeded constructs as described by Juncosa, N et al., 2006, Tissue Eng, 12:681-9. Accordingly, the use of different cell concentrations should be evaluated to examine a dosage dependent response in-vivo.

The above description and the drawings are provided for the purpose of describing embodiments of the invention and are not intended to limit the scope of the invention in any way. It will be apparent to those skilled in the art that various modifications and variations can be made without departing from the spirit or scope of the invention. Thus, it is intended that the present invention cover the modifications and variations of this invention provided they come within the scope of the appended claims and their equivalents.

What is claimed is:

1. A method of deriving isolated stem cells comprising:
implanting a matrix in a wound site of a living organism;
allowing cells to infiltrate the matrix;
removing the matrix containing the infiltrated cells from the wound site; and
removing the infiltrated cells from the matrix to provide isolated stem cells.

2. The method of claim 1, wherein the living organism is a mammal.

3. The method of claim 2, wherein the living organism is a human.

4. The method of claim 2, wherein the living organism is a mouse, rabbit, or rat.

5. The method of claim 1, wherein the matrix is an open cell sponge.

6. The method of claim 5, wherein the sponge is made of polyurethane.

7. The method of claim 1, wherein the isolated stem cells are positive for Oct-4 and SSEA-1 as determined by immunofluorescence staining.

8. The method of claim 7, wherein the isolated stem cells are negative for SSEA-3, SSEA-4, Tra-60, and Tra-80 as determined by immunofluorescence staining.

9. The method of claim 1, wherein the isolated stem cells have measurable telomerase levels while control fibroblasts have no measurable telomerase levels as determined by an enzyme-linked immunosorbent assay.

10. The method of claim 1, wherein the isolated stem cells respond to TGFβ to form capillary-like structures.

11. The method of claim 1, wherein the isolated stem cells are positive for Oct-4 and SSEA-1 as determined by immunofluorescence staining and have measurable telomerase levels while control fibroblasts have no measurable telomerase levels as determined by an enzyme-linked immunosorbent assay.

12. The method of claim 11, wherein the isolated stem cells are negative for SSEA-3, SSEA-4, Tra-60, and Tra-80 as determined by immunofluorescence staining.

13. The method of claim 11, wherein the isolated stem cells respond to TGFβ to form capillary-like structures.

14. The method of claim 12, wherein the isolated stem cells respond to TGFβ to form capillary-like structures.

15. The method of claim 1, wherein the isolated stem cells do not respond to acidic or basic FGF in a chemotaxis assay but do respond to PDGF-BB in a chemotaxis assay.

16. The method of claim 11, wherein the isolated stem cells do not respond to acidic or basic FGF in a chemotaxis assay but do respond to PDGF-BB in a chemotaxis assay.

17. The method of claim 12, wherein the isolated stem cells do not respond to acidic or basic FGF in a chemotaxis assay but do respond to PDGF-BB in a chemotaxis assay.

18. The method of claim 13, wherein the isolated stem cells do not respond to acidic or basic FGF in a chemotaxis assay but do respond to PDGF-BB in a chemotaxis assay.

19. The method of claim 14, wherein the isolated stem cells do not respond to acidic or basic FGF in a chemotaxis assay but do respond to PDGF-BB in a chemotaxis assay.

20. The method of claim 1, further comprising culturing the isolated stem cells.

21. The method of claim 1, further comprising purifying the isolated stem cells.

22. A method of treating wounds comprising:
implanting a matrix in a first wound site of a first living organism;
allowing cells to infiltrate the matrix;
removing the matrix containing the infiltrated cells from the wound site;
removing the infiltrated cells from the matrix to provide isolated stem cells; and
applying the isolated stem cells to a second wound site that is on the first or a second living organism.

23. The method of claim 22, wherein the second wound site is on the first living organism.

24. The method of claim 22, wherein the second wound site is on the second living organism.

25. The method of claim 22, wherein the second wound site comprises a tendon.

26. The method of claim 22, further comprising culturing the isolated stem cells before applying the isolated stem cells to the second wound site.

27. A method of deriving, culturing, and differentiating isolated stem cells comprising:
implanting a matrix in a wound site of a living organism;
allowing cells to infiltrate the matrix;
removing the matrix containing the infiltrated cells from the wound site;
removing the infiltrated cells from the matrix to provide isolated stem cells;
culturing the isolated stem cells in an undifferentiated state; and
subsequently differentiating the isolated stem cells into a specific cell type.
